(19) United States
(12) Patent Application Publication
(10) Pub. No.: US 2008/0020416 A1
(43) Pub. Date: Jan. 24, 2008

(54) Enzyme and Preparation Method

(75) Inventors: Mark Samuel Beaton McAlister, Cheshire (GB); Antonio Pineda-Lucena, Valencia (ES)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 10/598,280

(22) PCT Filed: Mar. 7, 2005
(86) PCT No.: PCT/GB05/00873

§ 371 Date: Jun. 12, 2007

(30) Foreign Application Priority Data
Mar. 10, 2004 (GB) 0405330.2

(51) Int. Cl. C12N 9/72; C12N 15/58; C12Q 1/37; C12N 15/62

(52) U.S. Cl. 435/23; 435/215

(57) ABSTRACT

A method for preparing a soluble protein comprising urokinase-type plasminogen activator (uPA) or an active fragment thereof, or a variant of either of these which has uPA activity, which method comprises contacting said protein with a buffer at a pH of from 8.5-10.5, said buffer comprising a reducing agent and an oxidising agent which forms a redox pair, wherein the reducing agent is present in excess compared to the oxidising agent, and wherein the reducing agent is present in a concentration of at least 5 mM. Material obtainable in this way forms a further aspect of the invention. It has been refolded in a “native-like” form and is useful in studies such as N.M.R. analysis to detect ligands.

The present invention relates to a method for producing an enzyme, specifically, urokinase-type plasminogen activator (uPA), which is particularly suitable for heteronuclear NMR studies or other biochemical, functional and structural studies as well as enzyme obtained by this method.

Urokinase-type plasminogen activator (uPA) is a serine protease involved in tumour metastasis and invasion. Inhibitors of uPA may have potential as drugs for prostate, breast and other cancers. uPA is a disulphide-bonded, multi-domain, glycoprotein of 411 residues, that is activated by plasmin to produce 2-chain uPA. Therefore the identification of ligands for uPA is an important target for pharmaceutical research.

Nuclear magnetic resonance (NMR) provides a method to monitor, at the amino acid and atomic levels, the structure and conformation of a protein in solution. The position of the signals in the spectra is extremely sensitive to the environment of the amino acids, and changes in the position of these signals can be correlated with interactions between the protein and another molecule.

EP-B-0866967, describes a technique whereby ligands to target biomolecules are identified using nuclear magnetic resonance (NMR). The approach relies on identification of amino acid residues that experience perturbation of chemical-shifts induced by binding of ligands to the protein and mapping of these chemical shift perturbations onto the three dimensional structure of the protein that has generally been solved previously by X-ray crystallography or by homology modelling. This approach requires protein samples comprising stable isotope labels (¹⁵N, ¹⁵N/²H, and/or ¹³C/²H). This technique is useful in identifying compounds that bind to the particular biomolecule, which can then act as leads in pharmaceutical research programmes. Thus it acts as method for structure-based inhibitor design by protein NMR (sometimes termd SAR-by-NMR).

Investigation of uPA using this method would therefore be desirable. Several different uPA constructs have yielded crystal structures in the literature (Spraggon, G., et al. (1995) Structure 3, 681-691; Neinaber, V. et al. (2000) J. Biol. Chem. 275, 7239-7248; Katz, B. et al. Chemistry and Biology (2000) 7, 299-312; Zeslawska, E. et al. (2000) J. Mol. Biol. 301, 465-475).

SAR-by-NMR approaches generally require large quantities (>100 mg) of uniformly ¹⁵N (²H) labelled protein. In order to identify which chemical shifts correspond to which amino acids in the protein, a sequence-specific assignment is generally required. Thus, triple resonance heteronuclear NMR experiments must be recorded that require uniformly ¹⁵N, ¹³C (²H) labelled protein in order to perform sequential resonance assignment.

A previous heteronuclear NMR study by Abbott labs, experts in the field of SAR-by-NMR (EP-B-0866967) relied on protein samples generated by partial ¹⁵N labelling methods based on expression in insect cells (Hadjuk et al., 2000). These studies yielded ¹⁵N-¹H HSQC spectra of poor quality consistent with partial, non-uniform biosynthetic labelling that would be of only limited use for SAR-by-NMR approaches to the study and optimisation of uPA inhibitors. This did not allow sequential assignment to be performed and therefore induced chemical shift perturbations measured in ligand binding experiments could not be interpreted directly with respect to the protein sequence.

The current state of the art generally allows uniform biosynthetic ¹⁵N, ¹³C (or any combination of these nuclei with ²H) labelling of proteins in only bacterial expression hosts. Multi-disulphide bonded proteins, such as uPA, are however generally expressed only in insoluble form in bacteria and therefore in order to support the above NMR experiments an efficient “refolding” method is required.

A method for refolding of uPA from inclusion bodies has previously been reported (Winkler et al., 1985) which was then later used to generate protein for successful protein structural studies by X-ray crystallography (Spraggon et al., 1985; Zeslawska et al., 2000). However, the protein production approach described by Zeslawska et al, did not yield sufficient quantities of native protein (equivalent to <10 μg native uPA per gram of wet cell pellet using LMW-uPA as control) to support stable isotope labelling for NMR studies.

There is therefore a need to produce uniformly stable isotope labelled uPA in sufficient quantity and quality to allow, for example, SAR-by-NMR to be carried out effectively.

According to the present invention there is provided a method for preparing a soluble protein comprising urokinase-type plasminogen activator (uPA) or an active fragment thereof, or a variant of either of these which has uPA activity, which method comprises contacting said protein with a buffer at a pH of from 8.5-10.5, said buffer comprising a reducing agent and an oxidising agent which forms a redox pair, wherein the reducing agent is present in excess compared to the oxidising agent, and wherein the reducing agent is present in a concentration of at least 5 mM.

The protein is suitably a modified form of urokinase-type plasminogen activator (uPA) or an active fragment thereof, or a variant of either of these which has uPA activity.

As used herein, the expression “modified form of urokinase-type plasminogen activator (uPA)” refers to non-native forms of the protein, which differ either because they are truncated, mutated or have proteins fused to them, and/or carry isotope labels. Examples of mutated proteins are proteins where one or more amino acids have been substituted for different amino acids, as well as deletion mutants where the deletions are either at the termini or are internal sequence deletions, or insertional mutants where one or more amino acids have been added to the sequence.

In particular, the protein is a non-native active fragment of urokinase-type plasminogen activator (uPA) or a variant thereof. Specific examples of such proteins are variants of a non-native truncated form or fragment of uPA, such as those described below. In particular, they are mutated in the N-terminal region. In addition, the proteins have a small number, for example up to 10, and preferably up to 5 amino acid substitutions.

The conditions described above, are more highly reducing, and at higher pH than conventionally used in refolding, provide an exceptionally good yield of high quality modified uPA or uPA type protein. In particular the protein obtained has been refolded so that it has a “native-like” three-dimensional structure and activity, in that it closely resembles the protein and activity found in nature.

The conditions are obtained by the use of the particular refolding buffer having the properties defined above.

The protein is suitably in uniformly stable isotope labelled form, which allows it to be used in, for example, NMR studies.

In particular, the buffer has a pH of from 9-10, and most suitably a pH of 9.5.

The redox pair suitable comprises a reduced and oxidised form of a reagent such as glutathione, cysteine or the like, as would be apparent to a skilled chemist. In particular the redox pair comprises reduced glutathione and oxidised glutathione. The reducing agent is present in a significant excess as compared to the oxidising agent. For instance, the ratio of reducing agent:oxidising agent is at least 5:1 and suitably in the range of from 5:1 to 15:1. A particular ratio of reducing agent:oxidising agent is about 10:1.

The concentration of reducing agent must also be quite high, being at least 5 mM, suitably from 8 mM-15 mM, and preferably about 10 mM.

A particularly preferred buffer for use in the method comprises 50 mM glycine, 10 mM reduced glutathione (GSH), 1 mM oxidised glutathione (GSSG).

Optionally it further comprises one or more additives selected from non-detergent sulphobetaine (NDSB 201), for example at 0.5-1M, and preferably at 1M, arginine such as L, D or D/L arginine or salts thereof, for example L-arginine hydrochlorides for example at 0.8-1.2M, such as 0.9M, L proline, for example at 0.8-1.2M, such as 1M, or 3-[{3-cholamidopropyl)dimethylammonio]1-propanesulfonate (Chaps) for example at 10-30 mM, such as 20 mM, or lauryl maltoside for example at 0.004-0.01% w/v, such as 0.006% w/v.

Preferably the additive is NDSB 201.

Protein obtained in this way allows generation of stable-isotope labelled samples of sufficient quality to allow execution of a full, robust SAR-by-NMR programme for uPA.

Preferably the protein is a modified form of human uPA, in particular an active fragment thereof, or a variant of any of these.

As used herein, the expression “variant” refers to proteins which have sequences of amino acids that differ from the base sequence from which they are derived (in this case native uPA, and preferably native human uPA) in that one or more amino acids within the sequence are substituted for other amino acids. Amino acid substitutions may be regarded as “conservative” where an amino acid is replaced with a different amino acid with broadly similar properties. Non-conservative substitutions are where amino acids are replaced with amino acids of a different type. Broadly speaking, fewer non-conservative substitutions will be possible without altering the biological activity of the polypeptide. Suitably variants will be at least 70% identical, more suitably at least 80% identical, for instance at least 90% identical, preferably at least 95% identical, and more preferably at least 98% identical to the base sequence.

Identity in this instance can be judged for example using the BLAST algorithm or the algorithm of Lipman-Pearson, with Ktuple:2, gap penalty:4, Gap Length Penalty:12, standard PAM scoring matrix (Lipman, D. J. and Pearson, W. R., Rapid and Sensitive Protein Similarity Searches, Science, 1985, vol. 227, 1435-1441).

The term “fragment thereof” refers to any portion of the given amino acid sequence which has the same enzymatic activity as the complete amino acid sequence. Fragments will suitably comprise at least 100 and preferably at least 200 consecutive amino acids from the basic sequence.

For instance, the method of the invention can be used to produce a fragment corresponding to amino acids 147-403, and preferably a fragment corresponding to amino acids 147-411 of the full length human uPA sequence as set out in Nagai et al., (1985) Gene 36, 183-188.7, and the numbering used for the sequence is as shown in this reference.

A particularly preferred protein for use in the method of the invention comprises a variant of such a fragment in which one or more modifications to the wild type sequence have been made in order to reduce or eliminate protease activity of the enzyme. For instance, it has been found that mutation of the serine residue found at position 356 of the wild type human uPA sequence to an amino acid other than serine, and in particular to alanine, can eliminate protease activity.

In addition, a particularly preferred protein of the invention has cysteine residues mutated so as to remove the disulphide bond that would otherwise tether the remaining A-chain peptide to the catalytic B-chain. In particular, cysteines at positions 148 and 279 of the wild type sequence are suitably mutated, for example to serine groups, so as to produce a product which is more amenable to SAR-by-NMR.

If desired also, residues can be added to the N-terminus of the protein, in particular a methionine and an alanine residue, as described by Zeslawska et at. (2000) supra. However, using the method of the invention, such additions are optional.

In particular, the protein used in the method of the invention comprises uPA or a fragment or variant thereof as defined above, which is fused to an amino acid sequence which is useful in purification of the protein. Particular examples of such sequences are tag sequences, such as “his tags”, which comprise at least 4 and suitably at least 6 consecutive histidine residues at a terminus of the protein, preferably the N-terminus. Alternatively other known purification sequences such as glutathione-S-transferase (GST sequences) can be fused to the uPA.

In particular, the protein construct purified for refolding using the invention is a protein of SEQ ID NO 1 or a variant thereof, and in particular a protein of SEQ ID NO 2.

SEQUENCE ID NO 1

| | |
|---|---|
| 1 | hhhhhhrsaq sgqktlrprf kiiggeftti |
| | enqpwfaaiy rrhrggsvty |
| 51 | vcggslispc wvisathcfi dypkkedyiv |
| | ylgrsrlnsn tqgemkfeve |
| 101 | nlilhkdysa dtlahhndia llkirskegr |
| | caqpsrtiqt iclpsmyndp |
| 151 | qfgtsceitg fgkenstdyl ypeqlkmtvv |
| | klishrecqq phyygsevtt |
| 201 | kmlcaadpqw ktdscqgdsg gplvcslqgr |
| | mtltgivswg rgcalkdkpg |
| 251 | vytrvshflp wirshtkeen glal |

SEQUENCE ID NO 2

| | |
|---|---|
| 1 | hhhhhhrsaq sgqktlrprf kiiggeftti |
| | enqpwfaaiy rrhrggsvty |
| 51 | vcggslispc wvisathcfi dypkkedyiv |
| | ylgrsrlnsn tqgemkfeve |
| 101 | nlilhkdysa dtlahhndia llkirskegr |
| | caqpsrtiqt islpsmyndp |
| 151 | qfgtsceitg fgkenstdyl ypeqlkmtvv |
| | klishrecqq phyygsevtt |
| 201 | kmlcaadpqw ktdscqgdsg gplvcslqgr |
| | mtltgivswg rgcalkdkpg |
| 251 | vytrvshflp wirshtkeen glal |

In this sequence, each letter is used in accordance with the conventional single letter code for amino acids.

If required, this protein construct is proteolytically cleaved, at a later stage in the purification, by plasmin (between K158 and I159) to produce I159-L411 that is ultimately used for NMR experiments.

The uPA used as starting material is suitably denatured prior to precipitation from the buffer, and this may be achieved, for example using denaturing reagents such as 8M urea or 6M guanidine hydrochloride (Gdn).

The protein used as a starting material is suitably recombinant uPA or an active fragment thereof, or a variant of any of these, which has been expressed in a transformed host cell, such as a eukaryotic or prokaryotic cell. In a particularly preferred embodiment, the uPA is expressed in a prokaryotic cell, and in particular, a bacterial cell such as E. coli. This allows high levels of protein to be obtained. The efficacy of the refolding scheme of the invention allows such material to be utilised in the preparation of high quality stable-isotope labelled material, which is suitable for SAR-by-NMR studies.

The protein may be recovered from inclusion bodies using conventional methods.

Specifically, the host cells such as the E. coli cells are transformed with a vector, which includes a nucleic acid sequence which encodes the desired protein. For instance, the nucleic acid may comprise the wild type uPA sequence as shown in (Nagai et al., 1985 supra.) or preferably a modified form of this which encodes an active fragment or variant of uPA as described above.

In a particularly preferred embodiment, at least some of the codons present in the wild-type sequence are modified so that they are optimised for expression in a bacterial cell. In particular, codons appearing at the beginning of the sequence, for example up to the first 20, more suitably up to the first 10 codons are optimised to bacterial, and preferably E. coli preference, as is understood in the art. This ensures that high levels of expression are achieved.

The expressed protein may then be recovered from inclusion bodies within the cultured cells, using conventional methods. In particular, the cells may be suspended in a diluent, in particular a buffer at about pH 8.0. A particular buffer solution comprises 50 mM NaH₂PO₄ and 0.3M NaCl. Optionally, proteases inhibitors may be included in the buffer at this stage, for instance EDTA-free protease inhibitor tablets (Roche, Inc.) may be added if required, to reduce protein loss as a result of protease activity.

Cells may then be lysed for example using an emulsifier, and separated for instance using a centrifuge. The solid residue remaining after supernatant and lipid layers are removed are then suitably resuspended, for instance in a buffer solution with a pH in the range of from 7.5-10.5, and suitably at about 8, optionally containing denaturing agents such as guanidine hydrochloride and/or urea. Alternatively, the buffer solution used at this stage, may, if desired, comprise the refolding buffer used in the method of the invention, which may optionally contain denaturing agents such as guanidine hydrochloride and/or urea.

The suspension is then incubated under suitable conditions to solubilise the inclusion body. Suitable conditions may include temperatures of 30° C. for a suitable period, for example of from 1 to 3 hours. The supernatant is then suitably removed, and any residue removed for instance by centrifugation to leave a protein solution.

Optionally, the solids remaining after removal of the supernatant may be subject to further resuspension/incubation steps to further enhance the yield.

If desired, the buffer used at this stage has a pH in the range of from 8.5-10.5, suitably about pH 9. Optionally, the protein can be refolded without further purification by contacting the protein with an appropriate refolding buffer as detailed below.

Preferably however, the solution is purified for example using column chromatography. The inclusion of a purification tag is useful in this context, as it means that the desired protein will bind to the column, until eluted with a suitable buffer. Suitable column materials and elution buffers would be apparent to a skilled biochemist. In particular, the column may be treated with a similar buffer to that used in the solution itself, followed by one or more buffers having progressively lower pH, for example down to 4.5, in order to elute the target protein. The buffer is suitably a denaturing buffer, for example containing urea, or guanidine hydrochloride, as described above.

Examples of suitable buffers are illustrated hereinafter as Buffers B, C and D.

Refolding of the purified protein present in the eluate is then suitably carried out by diluting it into the relatively high (8.5-10.5) pH buffer containing an excess of reducing agent as described above. Renaturation is suitably effected by a process of rapid dilution into a renaturing (refolding) buffer. Rapid dilution may be effected by pumping the solution of the protein at low flow rates for instance of about 0.1 ml/minute into a larger volume of a renaturing buffer with efficient mixing/stirring, such that the proportion of the volume of renaturing buffer is maintained at greater than ten-fold excess over the volume of protein solution added and preferably at more than one-hundred fold excess. Stirring may be continued over an extended period, for example of between 1 hour and 1 week, suitably from 2 days or more.

Subsequent concentration may be carried out using for example an ultrafiltration device, followed by dialysis with an activation buffer, for example pH 8.0. Any precipitate formed during dialysis is removed by centrifugation. The resultant solution contains the desired renatured protein, which can be separated from the residue, for example by column chromatography using for instance a benzamidine sepharose purification technique, and gel filtration. Particular examples of reaction conditions, which may be used, are illustrated hereinafter.

If desired or necessary, any product such as precipitate may be recycled by being denatured, for example using the denaturing agents described above, and refolded as described.

Using the method of the invention, it is possible to express a uPA construct at very high-levels in bacteria as insoluble inclusion bodies, and to purify, solubilise and efficiently refold the uPA construct in quantities sufficient for large-scale deuterium, ¹⁵N and ¹³C labelling. Recovery of yields of ˜5 mg protein from 50 g bacterial paste are possible using this method.

Thus in a particular aspect, the invention provides a method for preparing protein comprising uPA or an active fragment, or variant of any of these which has uPA activity, said method comprising transforming a bacterial host cell with a nucleic acid which encodes said protein, culturing transformed cells, isolating protein from inclusion bodies within the cells, denaturing the protein in solution in a buffer, and renaturing/refolding the protein in a buffer having a pH of from 8.5 to 9.5, said buffer comprising a reducing agent and an oxidising agent which forms a redox pair, wherein the reducing agent is present in excess compared to the oxidising agent, and wherein the reducing agent is present in a concentration of at least 5 mM.

Soluble, renatured proteins such as uPA obtainable using these methods forms a further aspect of the invention.

This renatured material can be biosynthetically labelled using conventional methods, and used in methods for identifying ligands for uPA using NMR as described in EP-B-00866967. In this method, NMR analysis of labelled protein in the presence of test compounds that are potential ligands for uPA is carried out. Alternatively other methods for identifying ligands such as isothermal titration calorimetry and differential scanning calorimetry as described for instance by Ladbury et al., ‘Biocalorimetry: Applications of calorimetry in the biological sciences’ (1996) (Edition 1) John Wiley & Sons Ltd, London, or Ward et al. Progress in Medicinal Chemistry (2001), 38:309-76, can be carried out on material obtained in this manner.

Alternatively the material obtained can be used in other biochemical, functional and structural studies including the production of crystals which can be used to solve the structure by X-ray crystallography. The invention will now be particularly described by way example with reference to the accompanying diagrammatic drawings in which:

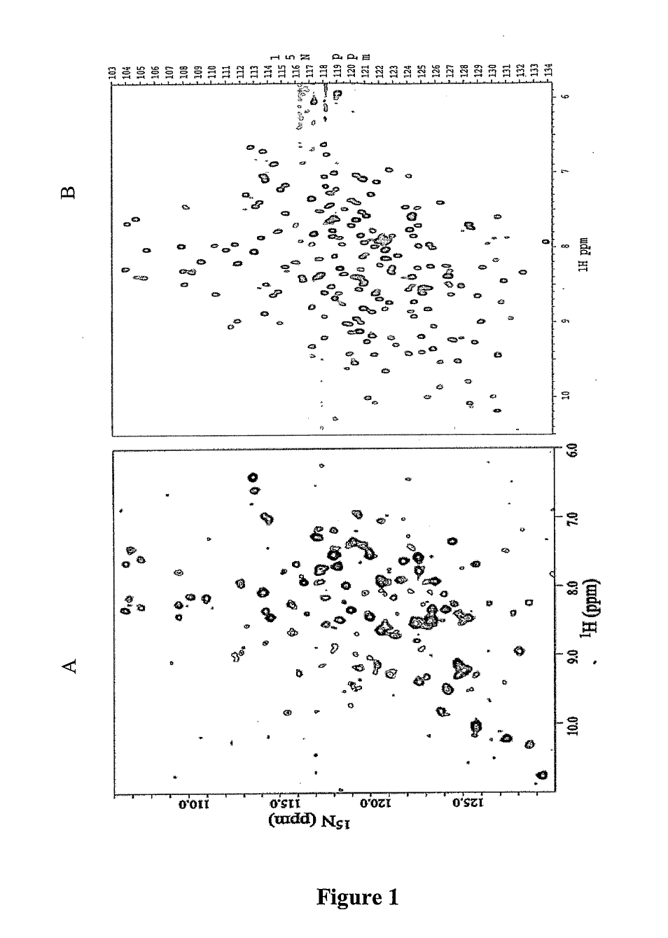

FIG. 1 shows a comparison of Nuclear Magnetic Resonance (NMR) spectra of uPA recorded by Abbott (left; Hajduk et al., J. Med. Chem., 43:3862-3866, 2000), with that obtained using uPA obtained by the method of the present invention (right). The y and x axes represent chemical shift in the nitrogen and proton dimensions, respectively, in ppm units.

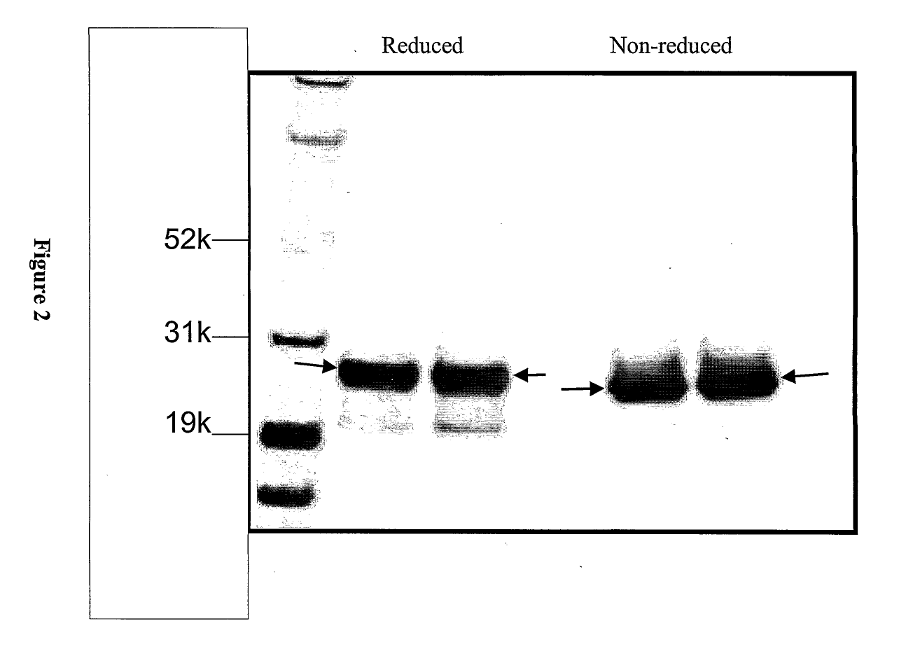

FIG. 2 shows by SDS-PAGE a comparison of activated, refolded uPA-AZ under reducing and non-reducing conditions. Samples of activated, refolded uPA-AZ (˜10 micrograms) were denatured by boiling in SDS-PAGE sample buffer under either reducing (20 mM DTT) or non-reducing (no DTT) conditions and duplicate samples were analysed on a 10% Bis-Tris Novex gel (Invitrogen, Inc) and stained with Coomassie Blue. This showed a single main band in both reduced and non-reduced lanes. The observed migration distance of the non-reduced samples was slightly longer (lower apparent molecular mass) than that of the reduced samples, consistent with the presence of intramolecular disulphide bonds. The absence of any higher apparent molecular weight bands in the non-reducing lanes indicated that intermolecular disulphide bonds were not present, suggesting that no mis-folded disulphide bonded aggregates were present.

In the following examples, the buffers described are summarised in the following table:

Buffers:

A. 50 mM NaH2PO4, 0.3 M NaCl pH 8.0.+8 tablets mini-complete (EDTA-free) protease inhibitors
B. 8 M urea, 0.1 M NaH2PO4, 0.01 M Tris.HCl, 10 mM b-mercaptoethanol pH 8.0
C. 8 M urea, 0.1 M NaH2PO4, 0.01 M Tris.HCl, 10 mM b-mercaptoethanol pH 6.3
D. 8 M urea, 0.1 M NaH2PO4, 0.01 M Tris.HCl, 10 mM b-mercaptoethanol pH 4.5.
E. 50 mM glycine, 10 mM reduced glutathione (GSH), 1 mM oxidised glutathione (GSSG), 1 M non-detergent sulphur betaine (NDSB 201), pH 9.5.
F. 15 mM Tris-HCl, 50 mM NaCl, pH 8.0
G. 50 mM Tris-HCl, 50 mM NaCl, pH 7.5
   All buffers were prepared immediately before use.

### EXAMPLE 1

uPA Cloning

uPA coding sequence was amplified by PCR from cDNA encoding human uPA. The construct generated in this study was a truncated form of human uPA encompassing the catalytic domain. This construct also had the following modifications with respect to the wild-type uPA sequence: MHHHHHHRSA. Codons were added to the 5′ end; C148S and C279A mutations were introduced by Quickchange mutagenesis and PCR respectively to remove a disulphide linkage; silent mutation of the first 6 codons encoding QCGQKT to codons of E. coli codon preference was achieved by PCR. This construct is hereafter referred to as uPA-AZ. This construct was designed so that on plasmin mediated proteolytic activation of uPA-AZ, a fragment comprising uPA159-411, C279A is generated that has previously been shown to yield crystals (Zeslawska et al., 2000). The oligonucleotide primers used for amplification of the uPA coding sequence were as follows:

5′ primer
GTCTCAGCAC TCGAGATCAG GTGTGACTGC GGATCCAGG
3′ primer
GTCTCAGCAC TCGAGTTAGA GGGCCAGGCC ATTCTCTT

The PCR product was then inserted into pCR-BluntII TOPO and the sequence was verified by DNA sequencing. The uPA coding sequence was then excised by digestion with BglII and XhoI and ligated with BamHI/XhoI digested pT73.3#6His to produce the final bacterial expression vector.

Expression in E. coli and Protein Purification.

6His-uPA147-411, C148S, C279A (uPA-AZ) was expressed in E. coli under the following conditions. BL21Star (DE3) cells transformed with the pT73.36His-uPA expression vector was cultured in LB medium containing 10 μg/ml Tetracyclin, at 37° C. In shake flask cultures expression was induced at OD600 nm ˜0.8 by addition of 1 mM IPTG and cultured for a further four hours before harvesting of the culture by centrifugation.

For high density fermentations the same transformed cell line was used. A seeder culture was prepared by transferring a 10 μl loopful of cells from the plate culture and inoculating it into 600 mls of M9 liquid medium containing 10 μg/ml of tetracycline, 2.0 g/L glucose and 1.0 g/L ¹⁵NH₄Cl, in a 2-litre Erlenmeyer flask. The culture was incubated at 37° C. on an orbital shaker at 250 rpm for 29 hours. A Braun Biostat C fermenter of working volume 30 litres, was charged with 20 litres of a defined minimal medium of the following composition in g/L: K₂SO₄, 1.0; MgSO₄.7H₂O, 0.75; H₃PO₄ (85%), 0.055; Na₂SO₄, 0.025; Glucose, 25.0; ¹⁵NH₄Cl, 10.0; Trace Elements (described below), 2 ml/litre; Thiamine hydrochloride, 0.008; FeSO₄.7H₂O, 0.025; AlCl₃.6H₂O, 0.2; CoCl₂.6H₂O, 0.08; H₃BO₄, 0.01; KI, 0.2; NiSO₄.6H₂O, 0.1; Na₂Mo₄.2H₂O, 0.5; ZnSO₄.7H₂O, 0.5; MnSO₄,0.379; CuCl₂.2H₂O, 0.02.

The seeder culture of 600 mls was inoculated into the prepared medium and maintained at 37° C. with aeration via a sparger at 0.5 vol/vol/min. The dissolved oxygen tension was maintained at 50% saturation by automatic control of the stirrer speed. The pH was maintained at 6.6 using 2M H₂SO₄ and 5M NaOH.

When the culture had reached an OD₅₅₀ₙₘ=5.0 expression of the uPA-AZ was induced by the addition of IPTG to give a final concentration of 0.4 mM. The process was continued for a further 8 hours until the biomass had reached an OD550ₙₘ=20. Cell paste was harvested by centrifugation in a chilled centrifuge and the cell paste was stored at ∓80° C. until extraction.

Expression of insoluble uPA-AZ was checked microscopically for the presence of inclusion bodies within the E. coli cells. The expression level as a percentage of the total microbial protein was determined by SDS-PAGE gel electrophoresis.

50 g of cell paste were thawed and resuspended in 500 ml of buffer A by homogenisation.

The cell suspensions was then lysed by passing twice through an Emulsiflex emulsifier, before spinning at 25,000 rpm, 30 mins. The supernatant was discarded and the lipid layer was gently scraped off the top of the pellet and discarded. The pellet was resuspended in fresh buffer A by homogenisation, before re-spinning at 25,000 rpm, 30 mins. The pellet was then resuspended in 200 ml of denaturing buffer B (˜5 ml/g wet pellet) and incubated at 30° C. in a water bath with occasional mixing for one hour to solubilise the inclusion body, before spinning at 25,000 rpm for 1 hour. The supernatant was decanted and then respun at 25 k rpm 30 mins before purifying as below

Purification:

Half of the above supernatant was loaded onto a 30 ml Ni-NTA column (XK26), pre-equilibrated in buffer B, before washing in 10 CV of buffer B then 10 CV of denaturing buffer C. The column was then inverted and uPA-AZ was eluted in 5 CV of denaturing buffer D. The other half of the supernatant was then processed as above and the eluates pooled. At this stage the eluate pool was 67 ml and A 280 nm=3.7 (˜2.55 mg/ml, therefore ˜170 mg of uPA-AZ in total). No uPA-AZ was observed in the column flow-through.

Refolding (Rapid Dilution)

The purified denatured uPA-AZ was spun at 45 k rpm, 30 mins in a 45Ti rotor in the ultracentrifuge to remove any traces of aggregated protein. The supernatant was then diluted ˜1/30 into 2000 ml of buffer E to give a final protein concentration of ˜100 μg/ml. Rapid dilution was achieved by pumping the protein solution at low flow rates (˜0.1 ml/min) into a 4 L beaker stirred rapidly on a magnetic stirrer at 4° C. Stirring was reduced after all protein had been transferred and the mixture was left at 4° C. for ˜5 days. A little precipitate was visible.

Activation:

The refolding mixture was concentrated by UF using a 10 k NMWL Pellican concentrator at 4° C. (˜12 hours), and then dialysed o/n against an activation buffer (buffer F above). Dialysis resulted in production of a large precipitate that contained ˜40% of the total protein. The dialysate was spun at 45 k rpm, 30 mins in a 45Ti rotor to remove insoluble protein and any aggregates.

1 μl of plasmin suspension (Roche) was added per ml of uPAf (1 mg/ml) and incubated at 4° C. overnight. As a result of this incubation, the protein construct was proteolytically cleaved, (between K158 and I159) to produce a fragment I159-L411 (activated uPA-AZ).

Benzanidine Sepharose Purification:

The solution was then loaded onto benzamidine-sepharose (Sigma) column (Vt=15 ml, XK16, pre-equilibrated in buffer F) and washed with buffer F until a flat baseline was obtained.

Activated uPA-AZ was eluted from the inverted column using 5 mM benzamidine in activation buffer. This was done in two batches.

Gel Filtration:

The activated uPA-AZ eluate peak fractions were pooled and loaded onto a Superdex 75 column 16/60 in two batches of ˜5 ml. The column was pre-equilibrated and run in buffer G. A single protein peak was observed.

Activated uPA-AZ peak fractions were then concentrated to the appropriate concentration 1-15 mg/ml before use or to 1-5 mg/ml before snap-freezing at and storage at −20° C.

uPA Activity Assays:

All uPA activity assays were performed using the SPECTROZYME UK assay (product no. 244, American Diagnostica Inc.) and found to have comparable activity to human LMW-uPA (product no. 125, American Diagnostica Inc.) (data not shown). Typically 0.5 volumes of chilled assay buffer (50 mM Tris HCl, pH 8.3) was mixed with 0.5 volumes of chilled substrate solution (0.2 mg/ml S-2444) and of chilled 0.5 ml of test sample before incubating at 20° C. Absorbance at 405 nm was then recorded using a spectrophotometer either at intervals or continuously.

Comparison of the activity of the refolded activated uPA-AZ with that of standards including commercially available LMW uPA (product no. 244, American Diagnostica Inc.) and the material produced as described in Katz et al. (2000) in a time-course assay showed almost identical activity indicating that the refolded material had essentially native levels of activity.

Characterisation of Refolded, Activated uPA:

SDS-PAGE analysis of the refolding mixture under reducing and non-reducing conditions suggests that essentially all of the protein is monomeric and disulphide bonded since only one band was observed for the non-reduced samples and no aggregate bands, and the non-reduced bands migrated at a slightly lower apparent molecular mass in comparison to the reduced bands as expected for a disulphide bonded protein (FIG. 2). The purified refolded uPA has been characterised by dynamic light scattering and gel-filtration and both analyses are consistent with a momomeric state as expected from the literature (data not shown). The observed mass for uPA (28399.0) obtained by ESI-mass spectrometry matched the expected mass (28398.12) for the activated uPA construct (uPA159-411, C148S, C279A) with 5 disulphide bonds to within 1 mass unit (data not shown).

### EXAMPLE 2

Nuclear Magnetic Resonance (NMR) Studies of uPA

NMR experiments on uPA were performed at 303 K on a Bruker Avance 600 MHz system equipped with a triple resonance (¹H/¹³C/¹⁵N) single-gradient 5 mm cryoprobe. Activated uPA-AZ samples were provided in 50 mM HEPES, pH 7.4, 50 mM NaCl. Prior to the NMR experiments, protein samples were extensively dialyzed using Amicon Ultra-15 centrifugal filter devices from Millipore (Billerica, Mass., USA), into the NMR buffer containing 50 mM HEPES, pH 7.3. Protein concentration was 0.1 mM. 5%(v/v) D₂O was added as a lock solvent

¹⁵N-¹H transverse relaxation-optimised spectroscopy-heteronuclear single-quantum correlation (TROSY-HSQC) (Pervushin et al., J. Biomol. NMR, 12:345-348, 1998) experiments, were recorded with evolution times of 85 milliseconds in the proton dimension and 25 milliseconds in the nitrogen dimension. The total acquisition time was 18 minutes. Data sets were processed with the program nmrPipe (Delaglio et al., J. Biomol. NMR, 6:277-293, 1995) and analyzed with the program SPARKY (Goddard and Kneller, University of California, San Francisco, USA).

The spectra obtained for uPA using this protocol were of very high quality and display the expected number of peaks for a protein of this size (see FIG. 1B), in contrast to the spectra recorded previously (see FIG. 1A). This is extremely important because it means that it is possible to monitor changes in any amino acid of the protein providing it interacts with a ligand.

In fact, the NMR assay has been found to be sensitive enough to detect changes in the environment of the protein in the presence of known inhibitors.

Another important advantage of uPA obtained by the method of the present invention is that it was possible to obtain sequential resonance assignments from triple-resonance heteronuclear NMR spectra acquired on samples of uPA uniformly labelled with ¹⁵N and ¹³C. This made it possible to identify the binding site of inhibitors by mapping the amino acid residues experiencing chemical shift perturbations onto the three-dimensional structure of uPA.

Furthermore, the NMR assay has been used in the identification of a number of novel inhibitors, and in the validation of hits from other screening methods.

1. A method for preparing a soluble protein comprising a modified form of urokinase-type plasminogen activator (uPA) or an active fragment thereof, or a variant of either of these which has uPA activity, which method comprises contacting said protein with a buffer at a pH of from 8.5-10.5, said buffer comprising a reducing agent and an oxidising agent which forms a redox pair, wherein the reducing agent is present in excess compared to the oxidising agent, and wherein the reducing agent is present in a concentration of at least 5 mM.

2. A method according to claim 1 wherein the protein is a non-native active fragment of urokinase-type plasminogen activator (uPA) or a variant thereof.

3. A method according to claim 1 wherein the protein is in uniformly stable isotope labelled form.

4. A method according to claim 1 wherein the buffer has a pH of from 9-10.

5. A method according to claim 4 wherein the buffer has a pH of 9.5.

6. A method according to claim 1 wherein the redox pair comprises reduced glutathione and oxidised glutathione.

7. A method according to claim 1 wherein the ratio of reducing agent:oxidising agent is at least 5:1.

8. A method according to claim 7 wherein the ratio of reducing agent:oxidising agent is in the range of from 5:1 to 15:1.

9. A method according to claim 8 wherein the ratio of reducing agent:oxidising agent is about 10:1.

10. A method according to claim 1 wherein the concentration of reducing agent is from 8 mM-15 mM.

11. A method according to claim 9 wherein the concentration of reducing agent is about 10 mM.

12. A method according to claim 1 wherein the buffer comprises 50 mM glycine, 10 mM reduced glutathione (GSH), 1 mM oxidised glutathione (GSSG).

13. A method according to claim 1 wherein the buffer further comprises one or more additives selected from non-detergent sulphobetaine (NDSB 201), arginine or salts thereof, L proline, 3-[{3-cholamidopropyl)dimethylammonio]1-propanesulfonate (Chaps) for example or lauryl maltoside.

14. A method according to claim 13 wherein the additive is non-detergent sulphobetaine (NDSB 201).

15. A method according to claim 1 wherein the urokinase-type plasminogen activator (uPA) is human uPA.

16. A method according to claim 1 wherein the protein is fused to an amino acid sequence which is useful in purification of the protein.

17. A method according to claim 16 wherein the protein comprises SEQ ID NO 2.

18. A method according to claim 1 wherein, in a preliminary step, the protein is denatured.

19. A method according to claim 18 wherein the denaturation is effected using 8N urea or 6M guanidine hydrochloride.

20. A method according to claim 16 or claim 17 wherein the protein product is subjected to a subsequent plasmin digestion step.

21. A method according to claim 1 wherein the protein is recombinant modified uPA or an active fragment thereof, or a variant of any of these, which has been expressed in a transformed host cell.

22. A method according to claim 21 wherein the host cell is a bacterial cell.

23. A method according to claim 22 wherein the protein is recovered from inclusion bodies in the host cell.

24. A method according to claim 22 or claim 23 wherein the host cell is transformed with a nucleic acid which encodes said protein, and wherein at least some of the codons present in the wild-type sequence of the nucleic acid are modified so that they are optimised for expression in a bacterial cell.

25. A method for preparing a soluble protein comprising uPA or an active fragment, or variant of any of these which has uPA activity, said method comprising transforming a bacterial host cell with a nucleic acid which encodes said protein, culturing transformed cells, isolating protein from inclusion bodies within the cells, denaturing the protein in solution in a buffer, and precipitating the protein from a buffer having a pH of from 8.5 to 9.5, said buffer comprising a reducing agent and an oxidising agent which forms a redox pair, wherein the reducing agent is present in excess compared to the oxidising agent, and wherein the reducing agent is present in a concentration of at least 5 mM.

26. A method according to claim 25 wherein the product is subjected to a plasmin digestion to form an active fragment of uPA.

27. Soluble protein comprising modified uPA or an active fragment, or variant of any of these which has uPA activity, obtainable by a method according to any one of the preceding claims.

28. Protein according to claim 27 which has been uniformly (≧98%) isotopically labelled with ¹⁵N and has a ¹⁵N-¹ H TROSY-HSQC NMR spectrum as shown in FIG. 1B, when measured in a buffer of 50 mM HEPES, pH 7.3 at a temperature of 303 K.

29. Protein according to claim 28 wherein the isotopic labelling comprises ¹⁵N or ¹³C or any combination of these nuclei with ²H.

30. A method for identifying ligands for uPA, said method comprising carrying out an analysis by NMR, isothermal titration calorimetry or differential scanning calorimetry on protein according to any one of claims 27 to 29, in the presence of test compounds, provided that in the case of NMR, the material is suitably labelled.

31. A method according to claim 30 for identifying ligands for uPA, said method comprising carrying out an analysis by NMR, wherein the protein is in uniformly stable isotope labelled form.

32. The use of protein according to any one of claims 27 to 29 for carrying out analysis by X ray crystallography.
